(19) United States
(12) Patent Application Publication
(10) Pub. No.: US 2020/0123497 A1
(43) Pub. Date: Apr. 23, 2020

(54) XENOBIOTIC-FREE CULTURE SYSTEM TO EXPAND HUMAN LIMBAL STEM CELLS

(71) Applicant: The Regents of the University of California, Oakland, CA (US)

(72) Inventors: Sophie Xiaohui Deng, Los Angeles, CA (US); Sheyla Gonzalez, Los Angeles, CA (US)

(73) Assignee: The Regents of the University of California, Oakland, CA (US)

(21) Appl. No.: 16/468,646

(22) PCT Filed: Dec. 12, 2017
(86) PCT No.: PCT/US2017/065869

§ 371 Date: Jun. 11, 2019

Related U.S. Application Data
(60) Provisional application No. 62/433,626, filed on Dec. 13, 2016.

(51) Int. Cl. C12N 5/00; C12N 5/074

(52) U.S. Cl. CPC C12N 5/0056; C12N 5/0607; C12N 2500/98; C12N 2500/35; C12N 2500/25; C12N 2500/46; C12N 2501/392; C12N 2501/11

(57) ABSTRACT

A human limbal epithelial stem xenobiotic free cell culture system is provided. The cell culture system typically includes a cell culture media comprising isoproterenol, Human Epidermal Growth Factor (EGF), N2 supplement, hydrocortisone, and an antibiotic. This cell culture media can efficiently propagate undifferentiated LSCs in the absence xenobiotic cells. These systems provide an optimized way to culture LSCs for use in human transplantation (e.g. in patients suffering from limbal stem cell deficiency) by minimizing the risk of cross-contamination and/or reagent toxicity to transplant recipients.

### REFERENCE TO RELATED APPLICATIONS

This application claims priority under Section 119(e) from U.S. Provisional Application Ser. No. 62/433,626, filed Dec. 13, 2016, entitled “XENOBIOTIC-FREE CULTURE SYSTEM TO EXPAND HUMAN LIMBAL STEM CELLS” by Sophie Xiaohui Deng et al., the contents of which are incorporated herein by reference.

### STATEMENT REGARDING FEDERALLY SPONSORED RESEARCH AND DEVELOPMENT

This invention was made with Government support under Grant Number 5P30EY000331 and R01EY021797, awarded by the National Institutes of Health. The Government has certain rights in the invention.

### TECHNICAL FIELD

This invention relates to cell culture media and systems, in particular cell culture media and systems for limbal stem cells that can be transplanted onto the cornea of patients suffering from limbal stem cell deficiency.

### BACKGROUND OF THE INVENTION

Limbal stem cell deficiency (LSCD) is a disorder characterized by the loss or dysfunctionality of limbal stem cells (LSCs) and its subsequent ability to regenerate the corneal epithelial surface. LSCD is characterized by persistent epithelial defects, conjunctivalization, neovascularization, scarring, and inflammation, all of which lead to corneal opacity, pain, photophobia, and ultimately blindness. Corneal transplantation is ineffective to treat severe to total LSCD because functional LSCs are not transplanted. The highest success rate of treating LSCD was achieved by Rama et al. in 2010 [1] by using single isolated LSCs cultured on 3T3 mouse fibroblasts feeder cells in a culture medium that contained fetal bovine serum (FBS). However, the presence of animal components in such xenobiotic culture systems poses a risk of transmitting animal diseases to human recipients after transplantation.

Supplemented hormonal epithelium medium (SHEM) is the conventional culture medium that provides an efficient growth of keratinocytes. However, components like cholera toxin and dimethyl sulfoxide (DMSO) within SHEM can be toxic after transplantation into humans. For example, DMSO enhances the permeability of the lipid cell membranes and Windrum et al. [12] reported DMSO's toxic effects after bone marrow stem cells transplantation. Additionally, though some studies have shown the importance of epidermal growth factor (EGF) in promoting LSC proliferation [13], EGF has also been shown by Wilson et al. [14] to increase LSC motility and decrease the induction of cytokeratin 12 expression. Furthermore, Miyashita et al. [16] showed that EGF decreases the survival of LSCs in the long term.

Different formulations of xenobiotic-free culture media that can sustain the growth of LSCs in vitro have been used, but their clinical success rates were reported to be lower than that using 3T3 feeder cells and bovine fetal serum [2-5]. Although some groups have reported successful LSC growth in xenobiotic-free conditions, it has been found that it is really challenging to both maintain the phenotype and expand the LSCs resembling the phenotype in vivo. Groups like Zakaria et al. [4, 5] and Sangwan et al. [2] have performed transplantations using xenobiotic-free culture systems. However, the expansion rate provided by these culture systems is low and inconsistent.

Accordingly, there is a need for culture mediums and systems that are optimized for use with limbal stem cells (LSCs) that are to be transplanted onto the cornea of patients suffering from limbal stem cell deficiency (LSCD). In particular, there is a need for an effective and complete xenobiotic-free culture system for culturing LSCs that minimizes the risk of cross-contamination and toxic effects when the LSCs are transplanted to the patient.

### SUMMARY OF THE INVENTION

The instant invention provides new human limbal epithelial stem cell (LSC) culture systems and materials and methods for making and using these systems. As illustrated by the technical data presented below, these systems include a cell culture media that provides for an efficient expansion of LSCs while maintaining the undifferentiated state of these LSCs. Embodiments of the cell culture media further include isoproterenol and relatively low concentrations of EGF, factors which eliminate the need for cholera toxin and DMSO. This cell culture media can efficiently propagate undifferentiated LSCs in the absence xenobiotic supplements. Consequently, these systems can provide an optimized way to culture LSCs for use in human transplantation (e.g. in patients suffering from limbal stem cell deficiency) by minimizing the risk of cross-contamination and/or reagent toxicity to transplant recipients.

The invention disclosed herein has a number of embodiments. One embodiment of the invention is a human limbal epithelial stem cell culture media comprising isoproterenol, Human Epidermal Growth Factor (EGF), and an antibiotic. In certain embodiments of the invention, the media is free of xenobiotic supplements. Typically, this media does not contain cholera toxin; and/or does not contain dimethylsulfoxide (DMSO). Optionally, the media comprises from 1% to 20% human serum (v/v), from 0.5-2 μg/mL isoproterenol, from 0.4-10 ng/mL Human Epidermal Growth Factor (EGF), from 0.4-5 μg/mL hydrocortisone, and/or at least one of penicillin, streptomycin, gentamicin or amphotericin B. In certain embodiments of the invention, the media comprises not more than 0.4 ng/mL Human Epidermal Growth Factor (EGF). Optionally, the cell culture media further comprises at least one of: insulin, transferrin, selenite, progesterone and putrescine. In certain embodiments of the invention, the media further comprises a denuded amniotic membrane. Typically, the media further comprises human limbal epithelial stem cells, for example human limbal epithelial stem cells are disposed within a limbal tissue explant. The human limbal epithelial stem cells growing in the media exhibit certain qualities that make them useful for transplantation, for example by comprising greater than 3% p63α bright cells.

Another embodiment of the invention is a method of growing human limbal epithelial stem cells comprising disposing the cells in a cell culture media disclosed herein (e.g. a culture media free of xenobiotic components) at a temperature (e.g. between 35° C. to 38° C.) and under CO₂ concentrations (e.g. between 4-10% CO₂) sufficient for the human limbal epithelial stem cells to grow. In typical embodiments of this invention, the methods are such that a preponderance of the human limbal epithelial stem cells do not differentiate after being disposed in the media. Typically in these methods, the conditions are controlled so that populations of human limbal epithelial stem cells growing in the media comprise greater than 3% p63α bright human limbal epithelial stem cells. In certain embodiments of these methods, the media comprises not more than 0.4 ng/mL Human Epidermal Growth Factor (EGF).

Another embodiment of the invention is a human limbal epithelial stem cell culture system. This cell culture system includes a cell culture media that typically comprises constituents of Dulbecco's Modified Eagle Medium as well as isoproterenol, Human Epidermal Growth Factor (EGF), hydrocortisone, and antibiotic agents. In certain embodiments, the media comprises DMEM/F12 medium, from 1% to 20% human serum (v/v), from 0.5-2 μg/mL isoproterenol, from 0.4-10 ng/mL Human Epidermal Growth Factor (EGF), from 0.4-5 μg/mL hydrocortisone, and penicillin, streptomycin, gentamicin and amphotericin B. Typically, the media does not contain cholera toxin and/or dimethylsulfoxide (DMSO). In typical embodiments of the invention, the concentration of human serum and/or EGF is controlled to modulate the state of cellular differentiation. Optionally for example, the concentration of human serum is not more than 10%, 9%, 8%, 7%, 6%, or 5% (v/v). While the media typically comprises 0.4-10 ng/mL Human Epidermal Growth Factor, optionally the media comprises not more than 1, 0.5 or 0.4 ng/mL Human Epidermal Growth Factor.

In the working embodiments of the invention that are disclosed below, these human limbal epithelial stem cells exhibit a certain phenotype and can, for example comprise greater than 3% p63α bright cells. In typical embodiments, the media further comprises a denuded amniotic membrane and includes human limbal epithelial stem cells (e.g. human limbal epithelial stem cells may be disposed within a limbal tissue explant). These culture systems and methods for producing transplantable human limbal stem cells can also utilize a new and efficient method for denuding amniotic membranes that are useful as substrates for culturing LSCs prior to transplantation into patients. In illustrative embodiments of the invention, a concentrated N2 supplement can be used to make the media, for example one that comprises 500 mg/L of insulin, 10,000 mg/L of transferrin, 0.52 mg/L of selenite, 0.63 mg/L of progesterone, and 1611 mg/L of putrescine, with this concentrated N2 Supplement being diluted 1/100 for use in the cell culture media.

Embodiments of the invention include methods of growing human limbal epithelial stem cells. The methods comprise disposing the cells in a system as described herein under conditions sufficient for the human limbal epithelial stem cells to grow (e.g. suitable temperature and CO₂ concentrations). In preferred embodiments, the human limbal epithelial stem cells do not differentiate. The differentiation state of the cell may be observed by observing cell morphology. The state of the cell may also be observed by observing expression of a polypeptide in the human limbal epithelial stem cells (e.g. p63α). The differentiation state of the cells may also be observed by using small cells as an index for undifferentiated cells (≤12 um in diameter). Other embodiments of the invention include a composition of matter comprising a cell culture media as disclosed herein.

Embodiments of the invention also include a transport system for cultured limbal stem cells. The transport vessel is designed, for example, to transport the cultured limbal stem cells (cLSCs, LSCs on the amniotic membrane carrier) from the cGMP manufacturing facility to the operating room where they will be transplanted. Embodiments of the transport vessel include a screw-cap and tight-sealed titanium container that has a ring attached to the lid to stabilize the cLSCs. The vessel is designed to maintain the cLSC graft stable down at the bottom and avoid substantial movements during transportation. The part of the container that makes this possible is the ring that is attached to the lid of the container. This piece can be either a solid ring or can be a broken ring with 3 spaces that will allow a better flow of the storage medium and will avoid spills.

Other objects, features and advantages of the present invention will become apparent to those skilled in the art from the following detailed description. It is to be understood, however, that the detailed description and specific examples, while indicating some embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

FIG. 1. Morphology (FIG. 1A), proliferation rate (FIG. 1B) and characterization (FIG. 1C) of LSCs cultured in four different CnT-PR conditions. Scale bar: 50 μm;

FIG. 2. Morphology (FIG. 2A), proliferation rate (FIG. 2B) and characterization (FIG. 2C) of LSCs cultured in different ESCM conditions. Scale bar: 50 μm;

FIG. 3. Morphology (FIG. 3A), proliferation rate (FIG. 3B) and characterization (FIG. 3C) of LSCs cultured in different SHEM conditions. Scale bar: 50 μm;

FIG. 4. Cell morphology (FIG. 4A), cell proliferation rate (FIG. 4B) and percentage of small cells (≤12 um) (FIG. 4C) among LSCs cultured in each base medium: CnT-PR, ESCM, and mSHEM. Scale bar: 50 μm; and

FIG. 5. Characterization of LSCs cultured in CnT-PR, ESCM, and mSHEM. Representative images of double immunostaining for the detection of K14-K12 (FIG. 5A), p63α (FIG. 5B) and PanK-Vim (FIG. 5C). Scale bar: 50 μm. Percentage of K14⁺/K12⁺ cells (FIG. 5D), p63α^{bright} cells (FIG. 5E), and PanK⁺/Vim⁺ cells (FIG. 5F).

FIG. 6. Diagram of an embodiment of a xenobiotic-free culture system for the expansion of human limbal epithelial cells. The diagram shows the vessel/container with the lid having a 20×20 mm denuded AM piece is then mounted on a filter paper ring for its stabilization and a 2×2 mm limbal explant.

FIG. 7. Transport vessel for the cLSCs. A. Diagram of the transport vessel showing two possible options for the stabilization ring attached to the lid. B. Photographs of the transport vessel showing stabilization ring design 1.

FIG. 8. Diagram with the specs of the transport vessel design. A. Measurements of the lid of the transport vessel. B. Measurements of the actual container of the transport vessel. Further machining is posteriorly made to finalize the stabilization ring and threating of the container.

### DETAILED DESCRIPTION OF THE INVENTION

In the detailed description of the invention, reference may be made to the accompanying drawings which form a part hereof, and in which is shown by way of illustration specific embodiments in which the invention may be practiced. It is to be understood that other embodiments may be utilized and structural changes may be made without departing from the scope of the present invention. A number of different publications are also referenced herein as indicated throughout the specification by reference numbers enclosed in brackets, e.g., [x]. A list of these different publications ordered according to these reference numbers can be found below in the section entitled “REFERENCES”. All publications, patents, and patent applications cited herein are hereby incorporated by reference in their entirety for all purposes.

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are generally well understood and commonly employed using conventional methodology by those skilled in the art. As appropriate, procedures involving the use of commercially available kits and reagents are generally carried out in accordance with manufacturer defined protocols and/or parameters unless otherwise noted.

In one aspect of the invention, a human limbal epithelial stem cell culture system is provided. The cell culture system comprises a cell culture media referred herein as a modified supplemented hormonal epithelium medium (mSHEM). The cell culture media or mSHEM provides an efficient expansion rate and maintains the undifferentiated state of LSCs with a very high efficiency. The only in vitro parameter shown to correlate with the clinical success is the percentage of p63α high-expressing cells (p63α bright cells) described by Rama et al. in 2010 [1]. Cultures with more than 3% of p63α bright cells are associated with successful transplantation in 78% of the patients. In illustrative experiments (see, e.g. EXAMPLES section below), the cell culture media has been shown to produce homogeneous cell outgrowths containing small and cuboidal limbal epithelial-like cells. Also, 100% of the outgrowths of LSCs cultured in the cell culture media contained a percentage of p63α bright cells higher than 3%.

The cell culture media is also typically completely xenobiotic-free. Since the human LSCs are not cultivated in the presence of animal components, the risk of cross-species contamination in clinical applications is eliminated.

The invention disclosed herein has a number of embodiments. One embodiment of the invention is a human limbal epithelial stem cell culture media comprising isoproterenol, Human Epidermal Growth Factor (EGF), and an antibiotic. In certain embodiments of the invention, the media is free of xenobiotic supplements. Typically, this media does not contain cholera toxin; and/or does not contain dimethylsulfoxide (DMSO). Optionally, the media comprises from 1% to 20% human serum (v/v), from 0.5-2 μg/mL isoproterenol, from 0.4-10 ng/mL Human Epidermal Growth Factor (EGF), from 0.4-5 μg/mL hydrocortisone, and/or at least one of penicillin, streptomycin, gentamicin or amphotericin B. In certain embodiments of the invention, the media comprises not more than 0.4 ng/mL Human Epidermal Growth Factor (EGF). Optionally, the cell culture media further comprises at least one of: insulin, transferrin, selenite, progesterone and putrescine. In certain embodiments of the invention, the media further comprises a denuded amniotic membrane. Typically, the media further comprises human limbal epithelial stem cells, for example human limbal epithelial stem cells are disposed within a limbal tissue explant. The human limbal epithelial stem cells growing in the media exhibit certain qualities that make them useful for transplantation, for example by comprising greater than 3% p62α bright cells.

Another embodiment of the invention is a method of growing human limbal epithelial stem cells comprising disposing the cells in a cell culture media disclosed herein (e.g. a culture media free of xenobiotic components) at a temperature (e.g. between 35° C. to 38° C.) and under CO₂ concentrations (e.g. between 4-10% CO₂) sufficient for the human limbal epithelial stem cells to grow. In typical embodiments of this invention, the methods are such that a preponderance of the human limbal epithelial stem cells do not differentiate after being disposed in the media. Typically in these methods, the conditions are controlled so that populations of human limbal epithelial stem cells growing in the media comprise greater than 3% p62α bright human limbal epithelial stem cells. In certain embodiments of these methods, the media comprises not more than 0.4 ng/mL Human Epidermal Growth Factor (EGF). In such methods, the differentiation state of the cell can observed by observing cell morphology and/or by observing expression of a polypeptide associated with the differentiation state in the human limbal epithelial stem cells.

Another embodiment of the invention is a human limbal epithelial stem cell culture system. In this context, a human limbal epithelial cell culture system refers to the components used to maintain and grow human limbal epithelial cells (e.g. containers, aqueous solutions). In certain embodiments, the cell culture system includes a cell culture media that typically comprises a plurality of constituents found in Dulbecco's Modified Eagle Medium: nutrient mixture F12 (DMEM/F12). Dulbecco's Modified Eagle Medium typically includes the following constituents/ingredients (mg/L): Calcium chloride dihydrate 265.000; Ferric nitrate nonahydrate 0.100; Magnesium sulphate anhydrous 97.720; Potassium chloride 400.000; Sodium chloride 6400.000; Glycine 30.000; L-Arginine hydrochloride 84.000; L-Cystine dihydrochloride 62.570; L-Glutamine 584.000; L-Histidine hydrochloride monohydrate 42.000; L-Isoleucine 105.000; L-Leucine 105.000; L-Lysine hydrochloride 146.000; L-Methionine 30.000; L-Phenylalanine 66.000; L-Serine 42.000; L-Threonine 95.000; L-Tryptophan 16.000; L-Tyrosine disodium salt 103.790; L-Valine 94.000; Choline chloride 4.000; D-Ca-Pantothenate 4.000; Folic acid 4.000; Nicotinamide 4.000; Pyridoxal hydrochloride 4.000; Riboflavin 0.400; Thiamine hydrochloride 4.000; i-Inositol 7.200; D-Glucose 4500.000; and Phenol red sodium salt 15.900. In addition to having one or more of these constituents, the media embodiments of the invention typically include isoproterenol.

In some embodiments of the invention, the media comprises DMEM/F12 medium, from 1% to 20% human serum (v/v). In certain embodiments, the media comprises from 0.5-2 μg/mL isoproterenol, from 0.4-10 ng/mL Human Epidermal Growth Factor (EGF), from 0.4-5 μg/mL hydrocortisone, and penicillin, streptomycin, gentamicin and amphotericin B. Typically, the media does not contain cholera toxin and/or dimethylsulfoxide (DMSO). In typical embodiments of the invention, the concentration of human serum and/or EGF is controlled to modulate the state of cellular differentiation. Optionally for example, the concentration of human serum is not more than 10%, 9%, 8%, 7%, 6%, or 5% (v/v). While the media typically comprises 0.4-10 ng/mL Human Epidermal Growth Factor, optionally the media comprises not more than 1, 0.5 or 0.4 ng/mL Human Epidermal Growth Factor.

In one or more embodiments, the cell culture media comprises constituents of a Dulbecco's Modified Eagle Medium: nutrient mixture F12 (DMEM/F12 medium), human serum, isoproterenol, human epidermal growth factor (EGF), N2 supplement, hydrocortisone, and an antibiotic compound. In certain embodiments, the human serum is provided in a range of 1% to 20% (v/v), the isoproterenol is provided in a range of 0.5-2 μg/mL, the human EGF is provided in a range of 0.4-10 ng/mL, and the hydrocortisone is provided in a range of 0.4-5 μg/mL. In certain embodiments, the media comprises not more than 0.5, 0.4, 0.3, 0.2 or 0.1 ng/mL human EGF. The antibiotic compound is at least one of penicillin, streptomycin, gentamicin or amphotericin B. In a preferred embodiment, the human serum is 5% (v/v), the isoproterenol is 1 μg/mL, the human epidermal growth factor (EGF) is 0.4 ng/mL, the N2 supplement is 1% (v/v), the hydrocortisone is 0.5 μg/mL, the penicillin-streptomycin is 100 units/mL and 100 μg/mL, respectively, and the gentamicin-amphotericin B is 0.01 mg/mL and 0.25 μg/mL, respectively. The certain instances, a concentrated N2 Supplement comprises 500 mg/L of insulin, 10,000 mg/L of transferrin, 0.52 mg/L of selenite, 0.63 mg/L of progesterone, and 1611 mg/L of putrescine. This concentrated N2 Supplement is typically diluted 1/100 for use in the cell culture media.

Notably, preferred embodiments of the cell culture media do not contain cholera toxin and/or dimethylsulfoxide (DMSO). Agents that increase the intracellular cyclic adenosine monophosphate (cAMP) levels have been used to increase cell proliferation in vitro. Isoproterenol is the most preferable for use in culture media. Ghoubay-Benallaoua et al. [7] proved that isoproterenol can substitute cholera toxin to enhance proliferation of LSCs. Judd et al. [17] also described different defined culture media formulations which support the in vitro cultivation of animal epithelial cells. These media comprise at least one agent that increases intracellular cAMP levels (preferably isoproterenol). In certain embodiments of the invention, the media further comprises human limbal epithelial stem cells. The human limbal epithelial stem cells may be disposed within a limbal tissue explant. In certain instances, the human limbal epithelial stem cells comprise greater than 3% p62α bright cells.

In another aspect of the invention, a method of growing human limbal epithelial stem cells is provided. The method comprises disposing the cells in a cell culture system as described herein at a temperature and under CO₂ concentrations sufficient for the human limbal epithelial stem cells to grow. In preferred embodiments, the human limbal epithelial stem cells do not differentiate. The differentiation state of the cell may be observed by observing cell morphology. The differentiation state of the cell may also be observed by observing expression of a polypeptide in the human limbal epithelial stem cells. For example, one can observe the state of differentiation by observing the expression of nuclear p63 expression and K14-K12 in the cytoplasm.

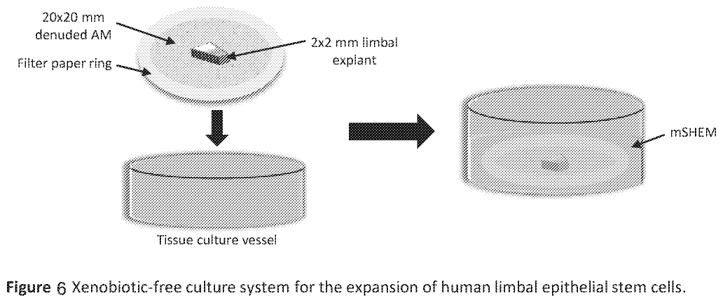

Furthermore, the xenobiotic-free manufacturing process to produce transplantable human limbal stem cells comprises human limbal epithelial stem cells (LSCs) expanded on denuded amniotic membrane (AM) from a limbal tissue biopsy. For the generation of the denuded AM, in one illustrative implementation, the epithelial cells from the AM are mechanically removed after a 2-hour ethylenediaminetetraacetic acid (EDTA) incubation at 37° C. and gentle scrapping, or by incubation with 125 μg/mL thermolysin for 1.5 minutes at 37° C. and 3 washes in PBS with vigorous shaking. A 4×4 cm denuded AM piece is then mounted on a filter paper ring for its stabilization (FIG. 6). The denuded AM piece is incubated in culture medium overnight at 37° C. and 5% CO₂. The next day, a limbal tissue explant piece of 2×2 mm is placed in the middle of the denuded AM piece. LSCs are cultured on the denuded AM for up to 21 days. The culture medium is changed every 1-3 days.

Embodiments of the invention also include a transport system for cultured limbal stem cells. The transport vessel is designed, for example, to transport the cultured limbal stem cells (cLSCs, LSCs on the amniotic membrane carrier) from the cGMP manufacturing facility to the operating room where they will be transplanted. Embodiments of the transport vessel include a screw-cap and tight-sealed titanium container that has a ring attached to the lid to stabilize the cLSCs. The vessel is designed to maintain the cLSC graft stable down at the bottom and avoid substantial movements during transportation. The part of the container that makes this possible is the ring that is attached to the lid of the container. This piece can be either a solid ring or can be a broken ring with 3 spaces that will allow a better flow of the storage medium and will avoid spills.

Embodiments of the invention also include a transport system for cultured limbal stem cells. Such embodiments include a transport system for cultured limbal stem cells comprising a transport container, a screw-cap that forms a tight seal with the transport container, a ring attached to the cap adapted to stabilize the cLSCs; and a cell culture media disclosed herein. The transport vessel is designed, for example, to transport the cultured limbal stem cells (cLSCs, LSCs on the amniotic membrane carrier) from the cGMP manufacturing facility to the operating room where they will be transplanted. Embodiments of the transport vessel include a screw-cap and tight-sealed titanium container that has a ring attached to the lid to stabilize the cLSCs. The vessel is designed to maintain the cLSC graft stable down at the bottom and avoid substantial movements during transportation. The part of the container that makes this possible is the ring that is attached to the lid of the container. This piece can be either a solid ring or can be a broken ring with 3 spaces that will allow a better flow of the storage medium and will avoid spills.

Further aspects and embodiments of the invention are disclosed in the following examples.

### EXAMPLES

### Example 1: Comparative Study of Xenobiotic-Free Media for the Cultivation of Human Limbal Epithelial Stem/Progenitor Cells

The culture of human limbal epithelial stem/progenitor cells (LSCs) in the presence of animal components poses the risk of cross-species contamination in clinical applications. We quantitatively compared different xenobiotic-free culture media for the cultivation of human LSCs. LSCs were cultured from 2×2 mm limbal tissue explants on denuded human amniotic membrane (AM) with different xenobiotic-free culture media: CnT-Prime supplemented with 0%, 1%, 5%, and 10% human serum (HS), embryonic stem cell medium (ESCM) alone or in combination with the standard supplemented hormonal epithelium medium (SHEM, control) at a 1:1 dilution ratio, and modified SHEM (mSHEM) in which cholera toxin and dimethyl sulfoxide (DMSO) were replaced by isoproterenol and the epidermal growth factor (EGF) concentration was reduced. Several parameters were quantified to assess the LSC phenotype: cell morphology, cell proliferation rate, cell size, outgrowth size, and expression of the undifferentiated LSC markers cytokeratin (K) 14, and p62α high-expressing (p63α^{bright}) cells, a mature keratinocyte marker K12, epithelial marker pancytokeratin (PanK) and stromal cell marker vimentin (Vim). Compared with SHEM, CnT-Prime base medium was associated with a lower proliferation rate and reduction in the proportion of stem cells generated regardless of the amount of HS supplemented (p<0.05); ESCM resulted in an increased proportion of PanK⁻/Vim⁺ stromal cells (p<0.05) and a decreased proportion of p63α^{bright} cells (p<0.05); mSHEM supported a similar proliferation rate (p>0.05), increased the number of small cells (diameter ≤12 μm; p<0.05), and provided a similar proportion of p63α^{bright} cells (p>0.05). Of the three base culture media evaluated, mSHEM was the most efficient and consistent in supporting the LSC growth.

Limbal stem cell deficiency (LSCD) is a corneal disorder in which corneal epithelial stem cells or limbal epithelial stem cells (LSCs) are absent or dysfunctional and, consequently, the ability to regenerate a corneal epithelial surface is lost. LSCD is characterized by persistent epithelial defects, conjunctivalization, neovascularization, scarring, and inflammation, all of which lead to corneal opacity, pain, photophobia, and ultimately blindness.

Corneal transplantation is ineffective to treat severe to total LSCD because functional LSCs are not transplanted. The highest success rate treating LSCD was achieved by using cultivated LSCs that were expanded from single LSCs cultured on 3T3 mouse fibroblasts feeder layers in culture medium that contained fetal bovine serum (FBS) [1]. However, the presence of animal components in the culture system posed the risk of transmitting animal diseases to human recipients of these LSCs.

Different formulations of a xenobiotic-free culture media that can sustain the growth of LSCs in vitro have been used, but their clinical success rates were reported to be lower than that using 3T3 feeder cells and bovine fetal serum [2-5]. For instance, transplantation of cultivated LSCs under xenobiotic-free explant culture supplemented with autologous serum achieved 71% clinical success [2]. Anatomical restoration of a corneal epithelial surface was achieved in 67% of patients who received cultivated LSCs using CnT-Prime (CnT-PR) medium (formerly CnT-20) supplemented with human serum (HS) in a Phase I/II trial [4,5]. Embryonic stem cell medium (ESCM) supplemented with KnockOut Serum Replacement has also been used to culture LSCs in vitro [6]. Supplemented hormonal epithelium medium (SHEM) has been further modified to replace cholera toxin with isoproterenol in vitro [7]. However, the efficiency of these different xenobiotic-free media in supporting the expansion of LSCs has not been compared directly using the same criteria.

It has been shown that the percentage of p62α high-expressing (p63α^{bright}) cells correlated with clinical success. In the present study, we investigated the efficiency of different xenobiotic-free culture media, with measures of efficiency defined as the cell proliferation rate and the relative amount of stem/progenitor cells including p63α^{bright} cells generated in each culture medium.

### Material & Methods

### Limbal Epithelial Stem Cell Culture

Human sclerocorneal tissues from 40- to 70-year-old donors were obtained from eye banks and handled in accordance with the tenets of the Declaration of Helsinki. The tissues were preserved in Optisol™ (Chiron Ophthalmics, Inc., Irvine, Calif.) or Life4C (Numedis, Inc., Isanti, Minn.) at 4° C. The death-to-preservation time was less than 12 hours, and the death-to-experiment time was less than 7 days. Amniotic membrane (AM) was selected as the culture substrate. Epithelial cells from the AM were mechanically removed as previously described [8] in an EDTA solution (Versene; Life Technologies, Carlsbad, Calif.).

To isolate limbal epithelial cell sheets, the iris, endothelium, conjunctiva and Tenon's capsule were removed from the sclerocorneal rim tissue. The rim was incubated in 2.4 U/mL of dispase II (Roche, Indianapolis, Ind.) at 37° C. for 2 h in DMEM/F-12 (Ham) medium (Life Technologies) followed by a gentle scrapping under the dissecting microscope. Single cells were obtained by incubation in 0.25% trypsin-EDTA (Life Technologies) for 5 minutes. Explant tissue pieces (dimensions, 2×2 mm) were also obtained from the limbal area. Experiments were performed with three to six donor tissues. LSCs were cultured by using limbal explants placed on denuded AM in different culture media (Table 1).

CnT-PR (CELLnTEC, Switzerland) was tested in the absence (CnT-PR 0) or presence of human serum (HS; Innovative Research, Novi, Mich.); the concentrations of

HS that were tested were 1% (CnT-PR 1), 5% (CnT-PR 5), and 10% (CnT-PR 10; Table 1). Embryonic stem cell medium (ESCM 1) was prepared as previously described [6] with knockout DMEM (Life Technologies) supplemented with 10% Knockout Serum Replacement (Life Technologies), 1% (v/v) N2 supplement (Life Technologies), 4 ng/mL of basic fibroblast growth factor (bFGF; Life Technologies), 1 mM L-glutamine, 0.1 mM β-mercaptoethanol (Life Technologies), and 1% (v/v) non-essential amino acids (Life Technologies). ESCM 2, which consisted of ESCM and SHEM at a 1:1 ratio (Table 1), was also tested.

The standard SHEM was prepared as previously described [9] and used as a control medium. Briefly, SHEM consisted of DMEM/F-12 medium supplemented with 5% HS, 1% (v/v) N2 supplement, 2.0 ng/mL of epidermal growth factor (EGF; Life Technologies), 8.4 ng/mL of cholera toxin (Sigma-Aldrich, St. Louis, Mo.), 0.5 μg/mL of hydrocortisone (Sigma-Aldrich), and 0.5% (v/v) of dimethyl sulfoxide (DMSO; Sigma-Aldrich).

Several modifications were made to SHEM (Table 1 and 2): SHEM1 was supplemented with 5% HS, and both cholera toxin and DMSO were not added to the medium. In SHEM 2, HS was increased to 10%. In SHEM 3, cholera toxin was replaced by isoproterenol7 (Sigma-Aldrich); the final concentration of isoproterenol was 1 μg/mL. In SHEM 4, DMSO and isoproterenol were added to the medium, but cholera toxin was not. Finally, in SHEM 5, the concentration of EGF was reduced to 0.4 ng/mL; the rest of the components and their concentrations were the same as those in SHEM 3.

Single LSCs on growth-arrested 3T3 mouse fibroblasts (the Howard Green laboratory, Harvard Medical School) cultured in unmodified SHEM with 5% fetal bovine serum (FBS; Life Technologies) served as the control (3T3 control) for all tissue donors tested. Explants on denuded AM (explant control) cultured in unmodified SHEM with 5% HS also served as another control of the LSC growth (Table 1). LSCs were cultured at 37° C. with 5% CO2 for up to 14 days. The medium was changed every 2-3 days.

### Analysis of Cell Proliferation Rate, Cells Size, Cell Morphology and Transplantability of Outgrowths

Cell proliferation or growth rate was calculated as the number of LSCs harvested divided by the number of LSCs seeded. Cell size was measured by Image J software (Colony Counter plugin). The percentage of cells whose diameter was ≤12 μm (small cells) was calculated for each culture condition.

Cell morphology of the outgrowths was assessed by using an inverted DMIL LED microscope (Leica Microsystems, Wetzlar, Germany). Cell images were taken with an Insight 11.2 color mosaic digital camera (Spot Imaging Solutions, Sterling Heights, Michigan). The outgrowth shape and size were measured; an outgrowth that was at least 13 mm in its smallest diameter after 10-14 days in culture was considered to be transplantable.

### Immunocytochemistry

Cultured LSCs were treated with trypsin to generate a single-cell suspension that was subjected to cytocentrifugation (Cytofuge; Thermo Scientific, Waltham, Mass.) onto glass slides (Fisher Scientific, Canoga Park, Calif.), and stored at −20° C. Cells were fixed in a 4% paraformaldehyde solution (Electron Microscopy Sciences, Hatfield, Pa.). Blocking and permeabilization were performed in a solution of 1% bovine serum albumin (Sigma-Aldrich) and 0.5% Triton X-100 (Sigma-Aldrich) for 30 minutes. Primary antibody incubation was done in a solution of 1% bovine serum albumin and 0.1% Triton X-100, overnight at 4° C. (antibodies are listed in Table 3). Secondary antibody incubation was done at room temperature for 1 hour. Nuclei were stained with Hoechst 33342 (Life Technologies) at room temperature for 10 minutes. Mounting was done with Fluoromount medium (Sigma-Aldrich). Pictures were taken with a Zeiss Image.A2 fluorescent microscope (Carl Zeiss Inc., Oberkochen, Germany) equipped with an Insight 11.2 color mosaic digital camera.

Cells that expressed high levels of p62α (p63α^{bright} cells) were quantified by using the Definiens Tissue studio software (Larchmont, N.Y.) as previously reported [10]. Quantitation of cells that expressed cytokeratin (K) 14, K12, pancytokeratin (PanK), or vimentin (Vim) was performed using Image J software.

### Statistical Analysis

Data were analyzed with the pairwise t-test. In the graphs, bars indicate the mean±standard error of the mean (SEM). P values <0.05 indicate statistical significance.

### Results

### Efficiency of Three Different Base Media in Supporting LSC Growth

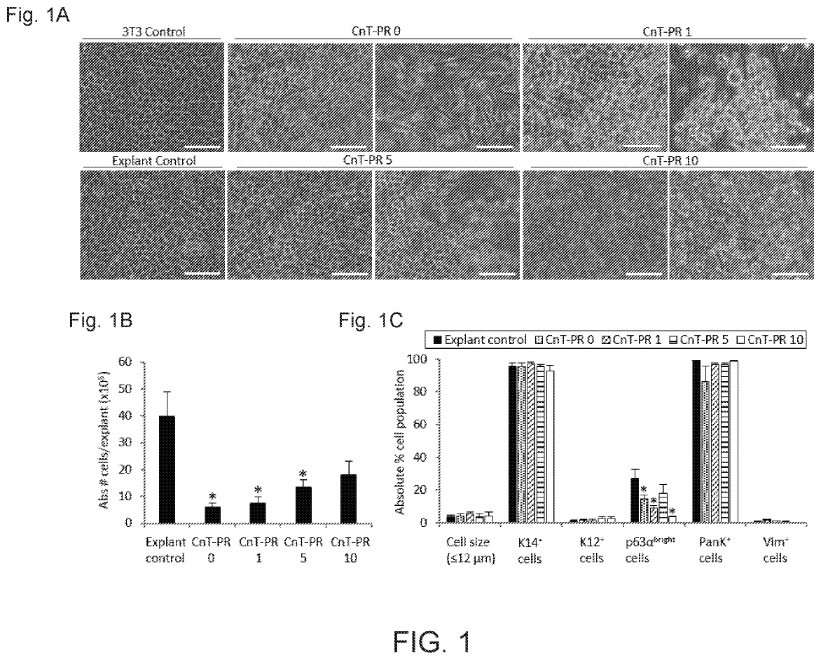

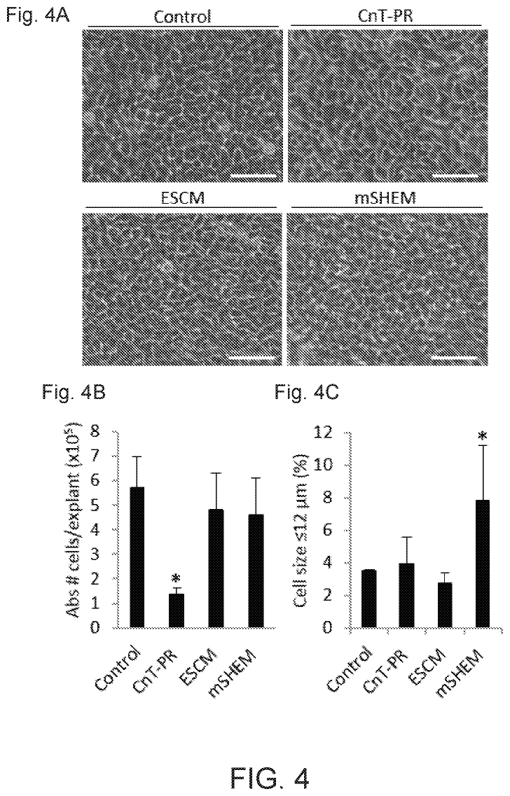

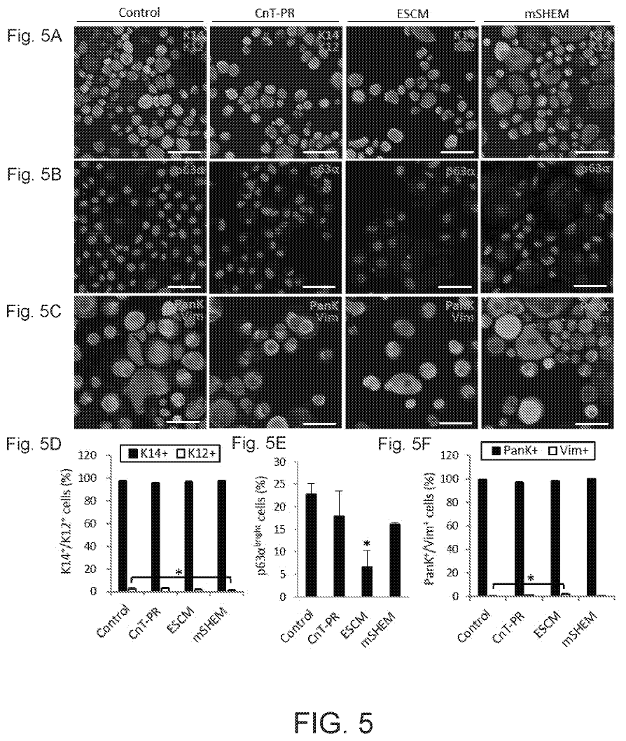

We first investigated the optimal formulation for each of the three base media: CnT-PR, ESCM, and SHEM (FIGS. 1-3 and Tables 1-2). After the optimal formulation of each base medium was determined, all three base media were compared to determine which one sustained the most efficient LSC growth (FIGS. 4 and 5, and Table 4).

Outgrowths of undifferentiated LSCs contained small and cuboidal epithelial-like cells in the control cultures: 3T3 control and explant control (FIG. 1A). LSC cultures in CnT-PR medium supplemented with different concentrations of HS showed variable and inconsistent results (FIG. 1). LSCs cultured in CnT-PR exhibited heterogeneous morphology; in some cases, the cells were spindle-shaped. A large quantity of floating dead cells and loose cell sheets at the edge were present. The increased concentration of HS in CnT-PR 5 appeared to reduce the quantity of floating dead cells and led to a more compact LSC morphology (FIG. 1A). The cell expansion rate was significantly reduced in all CnT-PR conditions (p<0.05), but the cell expansion rate in the CnT-PR culture with 10% HS (p=0.05) was comparable to that of the explant control in unmodified SHEM (FIG. 1B).

Analysis of the cell phenotype and quantitation of small cells (FIG. 1C) revealed that CnT-PR 5 was the most efficient of the CnT-PR-based media to culture LSCs. Compared to the explant control, the culture grown in CnT-PR 5 produced a similar proportion of small cells (≤12 μm; 3.5%±1.0% in the control vs. 3.9%±1.7%, p=0.84), a small proportion of differentiated K12⁺ cells (1.5%±0.4% in the control vs. 2.7%±1.1%, p=0.19), a high proportion of PanK⁺ epithelial cells (98.8%±0.4% in the control vs. 96.9%±0.9%, p=0.78), and a similar proportion of Vim⁺ stromal cells (1.1%±0.2% in the control vs. 1%±0.5%, p=0.50). The percentage of p63α^{bright} cells, which has been previously positively correlated with the transplantation success [1], was the highest in CnT-PR 5 among all CnT-PR conditions tested (p<0.05) and statistically comparable to the explant control (17.8%±5.7% vs. 27.6%±5.2%, respectively, p=0.25).

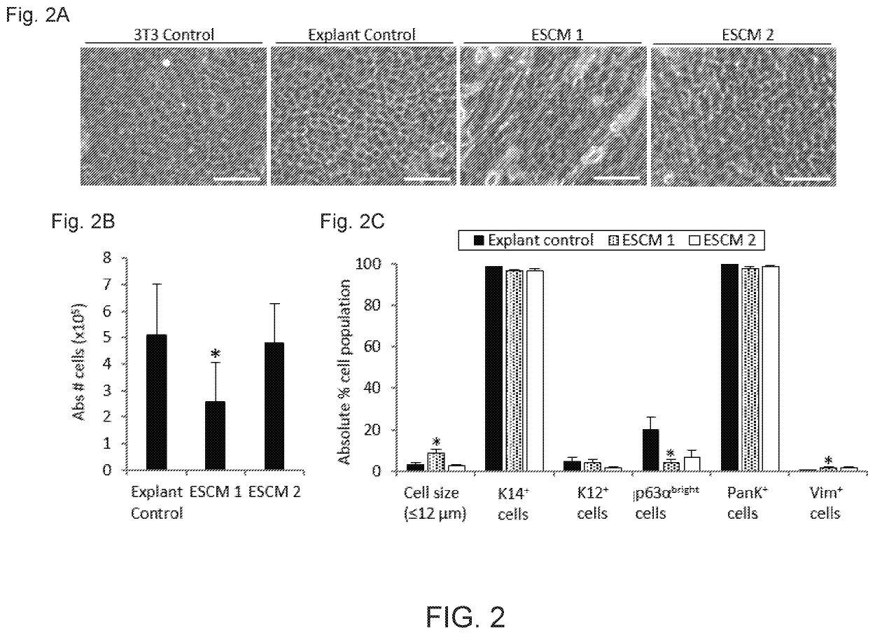

LSCs cultured in ESCM 1 had a heterogeneous morphology consisting of a mixture of small and cuboidal epithelial-like and fibroblast-like cells (FIG. 2A). LSC cultures in ESCM 2 shared a similar morphology as did those in the 3T3 control and explant control (FIG. 2A). The cell expansion rate in ESCM 1 was significantly lower than that in the explant control (49.7% slower in ESMC 1, p=0.01; FIG. 2B). In contrast to ESCM 1, ESCM 2 increased the LSC proliferation rate to a similar degree as in the explant control (p=0.81; FIG. 2B).

ESCM 1 increased the proportion of Vim⁺ stromal cells (1.8%±0.6% vs. 0.2%±0.1% in the explant control, p=0.04) and the percentage of small cells (8.4%±2.2% vs. 3.3%±0.9% in the explant control, p=0.04) and decreased the percentage of p63α^{bright} cells (4.4%±1.3% vs. 20.3%±5.7%, p=0.04; FIG. 2C). Compared to the explant control, ESCM 2, provided a statistically similar percentage of Vim⁺ cells (1.6%±0.8%, p=0.16), K12⁺ cells (1.8%±0.6%, p=0.06) and p63α^{bright} cells (6.7%±3.6%, p=0.06; FIG. 2C).

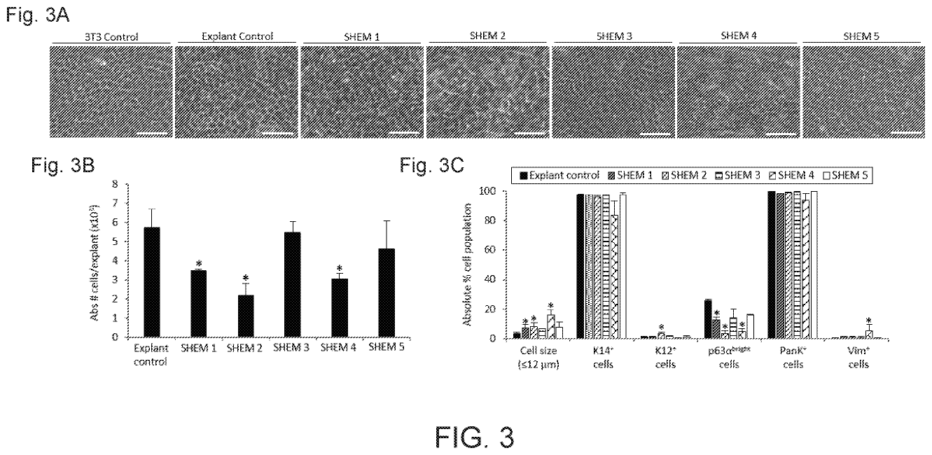

When SHEM was modified, the substitution of cholera toxin by isoproterenol in the absence of DMSO (SHEM 3 and SHEM 5) improved the cell sheet quality by reducing the amount of floating dead cells and the heterogeneous morphology seen in cultures grown in other SHEM formulations. SHEM 5, with its reduced concentration of EGF, provided cells with the best LSC morphology (FIG. 3A). The cell expansion rate was significantly reduced in most of the modified SHEM formulations (p<0.05) except SHEM 3 and SHEM 5 in which cholera toxin and DMSO were replaced by isoproterenol (both p≥0.05; FIG. 3B).

LSCs cultured in SHEM 3 and SHEM 5 showed a phenotype similar to that of cells grown in the explant control in terms of the percentage of small cells, p63α^{bright} cells, K12⁺ cells and Vim⁺ stromal cells (all p>0.05; FIG. 3C). However, other modifications of SHEM resulted in either a significant increase in the proportion of K12⁺ cells or Vim⁺ cells, or a decreased in the percentage of p63α^{bright} cells. Therefore, SHEM 5 appeared to be the best formulation among all 5 SHEM-based media.

### Growth of LSCs in the Different Optimized Base Media

As shown above, CnT-PR 5, ESCM 2 and SHEM 5 were the most efficient culture media for the growth of LSCs (highlighted in Table 1). These conditions were compared in a second set of experiments and will be referred to as CnT-PR, ESCM, and modified SHEM (mSHEM), respectively, for simplicity in the rest of the manuscript, FIGS. 4 and 5, and Table 4. Explant culture using the standard SHEM served as the control in these experiments and therefore will be referred to as the control.

We found that mSHEM supported the most compact and homogeneous cell outgrowths that contained small and cuboidal limbal epithelial-like cells (FIG. 4A). LSC cultures in CnT-PR and ESCM tended to be slightly more heterogeneous and variable (FIG. 4A).

ESCM and mSHEM supported a cell proliferation rate similar to that of the explant control (p>0.05; FIG. 4B). However, the LSC proliferation rate in CnT-PR was 76.6% reduced compared to the explant control (p=0.03; FIG. 4B).

A higher percentage of small cells (≤12 μm) was found in the mSHEM cultures than in other conditions including explant control (7.8%±3.4% vs. 3.5%±0.1%, respectively, p=0.04; FIG. 4C). Both CnT-PR and ESCM were able to produce a similar percentage of small cells as the explant control (3.9%±1.7% and 2.8%±0.6%, respectively; both p>0.05; FIG. 4C).

LSC outgrowths generated in each culture condition were classified as transplantable/non-transplantable on the basis of the size and shape of the cell sheet. Outgrowths that measured at least 13 mm in their smallest diameter were considered transplantable (Table 4). Cells grown in CnT-PR produced the smallest outgrowths and the smallest relative quantity of transplantable cell sheets (33.3% vs. 94.1% in the control, p=0.02; Table 4). In the ESCM cultures, the outgrowths were larger, and 62.5% were considered transplantable (p=0.03; Table 4). In comparison with ESCM and CnT-PR, mSHEM produced the most homogeneous outgrowths, and 100% were considered to be transplantable. On average, both diameters of cell outgrowths in mSHEM exceeded the 13-mm requirement (Table 4).

We next characterized the cell population in the cultures. The expression of K14 and K12 protein was analyzed to distinguish the proportion of undifferentiated corneal epithelial cells from the differentiated ones. Outgrowths from all three conditions contained a similar percentage of K14⁺ cells, which was >95% (p>0.05; FIG. 5A and D). Only cultures grown in mSHEM contained a significantly smaller proportion of differentiated K12⁺ cells (1.3%±0.5% vs. 2.5%±1.2% in the control, p=0.01). Cultures grown in CnT-PR contained 2.7%±1.1% K12⁺ cells, and cultures grown in ESCM contained 1.8%±0.6% K12⁺ cells (p>0.05; FIG. 5A and D). Overall, such small proportions of differentiated K12⁺ cells are not clinically significant.

We measured the percentage of p63α^{bright} cells in the cultures as a potential indication of the LSC transplantation success [1]. Cultures grown in CnT-PR and mSHEM contained proportions of p63α^{bright} cells (17.8%±5.7% and 16.1%±0.4%, respectively) similar to those in the explant control (22.8%±2.4%, p>0.05; FIG. 5B and E). ESCM was the culture condition that produced the least amount of p63α^{bright} cells (6.7%±3.6%, p=0.04; FIG. 5B and E).

The expression of PanK and Vim proteins was examined to determine the proportion of epithelial cells (PanK⁺/Vim⁻ and PanK⁺/Vim⁺) and stromal cells (PanK³¹ / Vim⁺) in the cultures. In general, all the cultures had a high percentage of either PanK⁻/Vim⁻ or PanK⁺/Vim⁺ cells (>95% in all the conditions, p>0.05; FIG. 5C and F). Only the ESCM cultures contained a proportion of PanK⁻/Vim⁺ stromal cells (1.6%±0.8%) that was significantly larger than that of the control (0.6%±0.3%, p=0.04).

### Discussion

To have a standardized LSC therapy using cultivated LSCs, there is the need for an optimized and xenobiotic-free culture system for such grafts. In the present study, we investigated three different base culture media in their ability to support the expansion of LSCs using a standard set of quantifiable criteria. The optimal formulation of each of the three base medium was determined before these three base media were directly compared with one another.

CnT-PR appears to be less efficient in supporting the growth of LSCs in vitro than the standard SHEM. We hypothesized that the LSC growth efficiency could be improved by supplementing a higher level of HS. We found that CnT-PR supplemented with 5% HS (CnT-PR 5) was better than CnT-PR supplemented with 1% HS in maintaining the LSC phenotype of most LSC cultures. However, the CnT-PR 5 medium was still less efficient than the standard SHEM in supporting the expansion of the LSC population.

LSCs and limbal stromal niche cells have been cultured as spheres in 3D Matrigel® using ESCM to prevent differentiation and to maintain the clonal growth of LSCs [6]. This method was excellent in maintaining the close association between LSCs and limbal stromal cells, but LSC proliferation was not optimal [6]. This finding is consistent with our own: when ESCM alone was used, the LSC proliferation rate was low, and some areas of outgrowth contained spindled, fibroblast-like cells. ESCM may favor the growth of these fibroblast-like stromal cells over the epithelial cells; this possibility is supported by the increase in the number of small, stromal-like PanK⁻/Vim⁺ cells. In a 3D system such as Matrigel®, the association between stromal cells and LSCs helped in the maintenance of LSC phenotype, which is similar to the effect of the in vivo niche [6]. However, in a 2D system in which both LSCs and stromal cells are plated together on a culture dish (e.g., a 2D system such as an explant culture on AM), there might be competition for the growth space and growth factors that favor the growth of stromal cells over the epithelial cells.

The modified SHEM without DMSO and cholera toxin provided the most efficient and consistent growth of LSCs. Isoproterenol is a non-selective beta-adrenergic agonist that has been previously shown, at a concentration of 1 μg/mL, to efficiently replace cholera toxin and thus enhance cell proliferation [7]. DMSO enhances the permeability of the lipid cell membranes during the cell culture period [11]; however, when DMSO has been included in stem cell cultures used for bone marrow stem cell transplantation, there have been reports of toxic side effects such as cardiovascular and respiratory issues due to DMSO's dose-dependent vasoconstrictor effect [12]. To minimize the potential toxicity of DMSO, different cell culture strategies were evaluated; among all conditions tested, the removal of DMSO in combination with the addition of isoproterenol resulted in the most efficient LSC growth. This strategy would signify a safer way to culture the LSCs for future transplantation by potentially diminishing the potential toxic effects of DMSO and cholera toxin to the recipient.

EGF appears to have different effects on LSCs in culture. Some studies have shown the importance of EGF in promoting LSC proliferation [13]; other studies have found an increase in LSC motility and a decrease in the induction of K12 expression in the presence of EGF [14]. EGF has also been shown to increase survival and colony-forming efficiency, but not necessarily the LSC growth rate [15]. Moreover, long-term cultures in the presence of EGF have not survived more than 3 months [16]. When EGF concentration in cultures was reduced five times from 2.0 ng/mL to 0.4 ng/mL, we found a more homogenous LSC-like cell morphology and a slight increase in the percentages of small cells and p63α^{bright} cells without a significant decrease in the proliferation rate. Therefore, short-term use of reduced EGF concentrations may favor cell survival and maintain the same degree of proliferation.

In conclusion, a robust xenobiotic-free culture system that can consistently support a sufficient expansion and maintain the undifferentiated state of LSCs is highly desired to achieve a successful reconstruction of a normal corneal epithelial surface in eyes with severe or total LSCD. Herein, we present a comparative analysis on different xenobiotic-free culture media using a set of quantitative criteria to standardize the cultivated LSCs characterization. A modified SHEM-based xenobiotic-free medium can consistently support LSC expansion from different limbal donor tissues.

### Tables

TABLE 1
Media conditions tested.
Media conditions

| | ESCM | |
|---|---|---|
| CnT-PR (Explant) n = 4 | (Explant) n = 6 | SHEM (Explant) n = 3 |
| CnT-PR 0 | ESCM 1 (ESCM) | SHEM 1 |
| (CnT-PR w/o HS) | | (SHEM −Chol −DMSO) |
| CnT-PR 1 | ESCM 2 | SHEM 2 |
| (CnT-PR 1% HS) | (SHEM:ESCM) | (SHEM10 −Chol −DMSO) |
| CnT-PR 5 | | SHEM 3 (SHEM −Chol |
| (CnT-PR 5% HS) | | −DMSO +Ipr) |
| CnT-PR 10 | | SHEM 4 (SHEM −Chol |
| (CnT-PR 10% HS) | | +DMSO +Ipr) |
| | | SHEM 5 (SHEM −Chol |
| | | −DMSO +Ipr +1XEGF) |

3T3 Control
Explant Control
Chol: cholera toxin;
CnT-PR: CnT-Prime,
epithelial: culture medium;
DMSO: dimethyl sulfoxide;
EGF: epithermal growth factor;
ESCM: embryonic stem cell medium;
Expl: explant;
FBS: fetal bovine serum;
HS: human serum;
Ipr. isoproterenol/Isuprel;
SHEM: supplemented hormonal epithelium medium;
SCs: single cells.

TABLE 2
SHEM media composition

| | HS | EGF | DMSO | Chol | Ipr |
|---|---|---|---|---|---|
| Explant Control | 5% | 2.0 ng/mL | Yes | Yes | No |
| SHEM 1 | 5% | 2.0 ng/mL | No | No | No |
| SHEM 2 | 10% | 2.0 ng/mL | No | No | No |
| SHEM 3 | 5% | 2.0 ng/mL | No | No | Yes |
| SHEM 4 | 5% | 2.0 ng/mL | Yes | No | Yes |
| SHEM 5 | 5% | 0.4 ng/mL | No | No | Yes |

Chol: cholera toxin;
DMSO: dimethyl sulfoxide;
EGF: epithermal growth factor;
HS: human serum;
Ipr: isoproterenol/Isuprel;
SHEM: supplemented hormonal epithelium medium.

TABLE 3
Primary antibodies used for immunocytochemistry.

| Primary Antibody | Dilution | Source and Catalogue # |
|---|---|---|
| K12 | 1:100 | Santa Cruz Biotechnology sc-25722 |
| K14 | 1:50 | Fisher Scientific MS-115-R7 |
| p63α | 1:100 | Cell Signaling Technology #4892 |
| PanK | 1:100 | DAKO |
| Vim | 1:100 | Abcam ab |

K12: cytokeratin 12;
K14: cytokeratin 14;
PanK: pancytokeratin;
Vim: vimentin.

TABLE 4
Size and transplantability of the outgrowths
in the different media conditions.

| | Major diameter | Minor diameter | Transplantable |
|---|---|---|---|
| | (mm) | (mm) | outgrowths (%) |
| Control | 26.9 ± 3.9 | 21.4 ± 1.7 | 94.1 |
| CnT-PR | 13.5 ± 3.2 | 11.0 ± 2.5 | 33.3 |
| ESCM | 27.3 ± 2.9 | 13.2 ± 2.0 | 62.5 |
| mSHEM | 25.0 ± 0.0 | 23.0 ± 2.0 | 100.0 |

### Example 2: Transport System for the Cultured Limbal Stem Cells

This example describes a transport vessel designed to transport the cultured limbal stem cells (cLSCs, LSCs on the amniotic membrane carrier) from the cGMP manufacturing facility to the operating room where they will be transplanted.

The transport vessel for the cLSCs is a screw-cap and tight-sealed titanium container that has a ring attached to the lid to stabilize the cLSCs (see, e.g. FIGS. 6-8). This was developed at the Machine Shop of SEI (UCLA).

The vessel is made from titanium 6AL4V or 6AL4V ELI alloys that contains 6% Aluminum and 4% Vanadiumor (Grade 23). These are the most common types of titanium used in medicine. This titanium grade has less oxygen so it is less corrosive than other titanium grades and non-leachable.

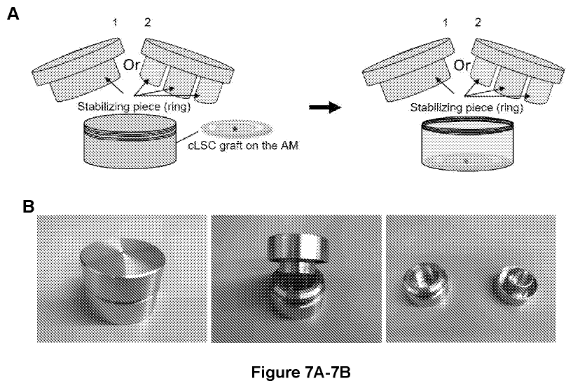

The vessel is designed to maintain the cLSC graft stable down at the bottom and avoid substantial movements during transportation (see, e.g. FIG. 7). The part of the container that makes this possible is the ring that is attached to the lid of the container. This piece can be either a solid ring (see, e.g. Design 1 in FIG. 7A) or can be a broken ring with 3 spaces that will allow a better flow of the storage medium and will avoid spills (see, e.g. Design 2 in FIG. 7A). Photographs of the actual transport vessel with design 1 can be seen in FIG. 7B.

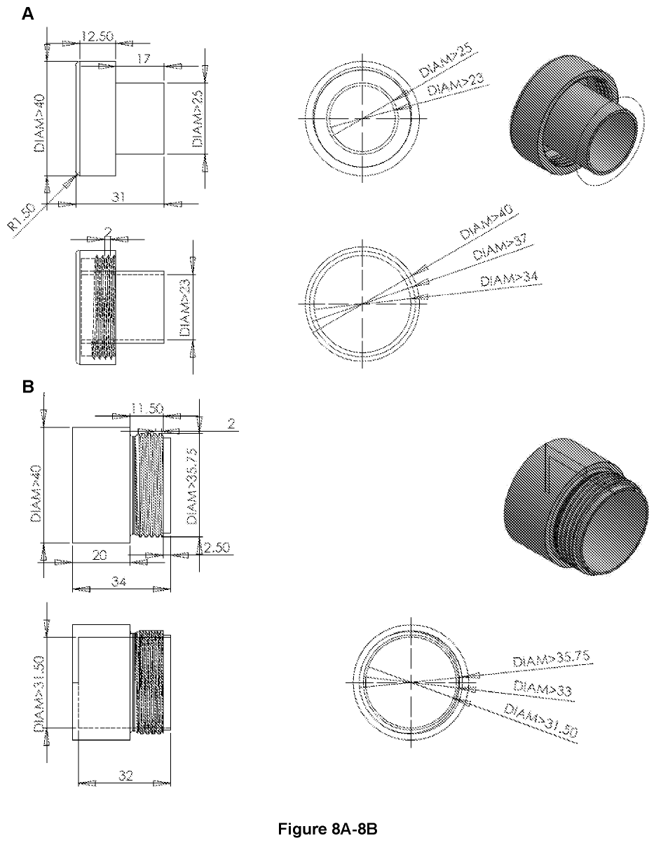

The dimensions of an illustrative embodiment of the container are specified in FIG. 8. Dimensions of the transport vessel are susceptible to be slightly modified.

The cLSCs will be stored in the storage medium which can have a composition of the culture medium disclosed herein (see, e.g. Table 5).

TABLE 5
Storage medium components.

| Components | Final concentration | Range concentration |
|---|---|---|
| DMEM/F12 | 1:1 (v/v) | — |
| CTS N2 | 1% (v/v) | — |
| | 0.01 mM transferrin | |
| | 0.86 uM insulin | |
| HEPES | 0.015 M | — |
| Human EGF | 0.4 ng/mL | 0.4-10 ng/mL |
| Human AB serum | 1% (v/v) | 1%-10% (v/v) |
| Hydrocortisone | 0.5 ug/mL | 0.4-5 μg/mL |
| Penicillin-Streptomycin | 1% (v/v) | — |
| | 100 units/mL Penicillin | |
| | 100 ug/mL streptomycin | |
| Gentamicin | 0.01 mg/mL | — |
| Amphotericin B | 0.25 ug/mL | — |

In embodiments of the invention, temperature during transportation can be between 17-22° C., and can be monitored by using an USB temperature data logger that uses a FDA 21CFR11 compliant software. A small and low-temperature incubator that can be set at a defined temperature will be used.

CTS N2 refers to reagents such as Cell Therapy Systems N-2 CTS™ (100X) Supplement (Catalog number: A1370701), which is a serum-free supplement for the growth and expression of post-mitotic neurons and tumor cells of neuronal phenotype. Cell Therapy Systems N-2 CTS™ (100X) supplement is a chemically defined, 100X concentrate of Bottenstein's N-1 formulation (see, e.g. Bottenstein, J. E. (1985) Cell Culture in the Neurosciences, Plenum Press: New York and London).

### REFERENCES

This description references a number of different publications as indicated throughout the specification by reference numbers enclosed in brackets, e.g., [x]. A list of these different publications ordered according to these reference numbers can be found below.
[1] Rama, P., et al. Limbal stem-cell therapy and long-term corneal regeneration. N Engl J Med 363, 147-155 (2010).
[2] Sangwan, V. S., et al. Clinical outcomes of xeno-free autologous cultivated limbal epithelial transplantation: a 10-year study. Br J Ophthalmol 95, 1525-1529 (2011).
[3] Shortt, A. J., et al. Three-year outcomes of cultured limbal epithelial allografts in aniridia and Stevens-Johnson syndrome evaluated using the Clinical Outcome Assessment in Surgical Trials assessment tool. Stem Cells Transl Med 3, 265-275 (2014).
[4] Zakaria, N., et al. Standardized limbal epithelial stem cell graft generation and transplantation. Tissue Eng Part C Methods 16, 921-927 (2010).
[5] Zakaria, N., et al. Results of a phase I/II clinical trial: standardized, non-xenogenic, cultivated limbal stem cell transplantation. J Transl Med 12, 58 (2014).
[6] Xie, H. T., Chen, S. Y., Li, G. G. & Tseng, S. C. Isolation and expansion of human limbal stromal niche cells. Invest Ophthalmol Vis Sci 53, 279-286 (2012).
[7] Ghoubay-Benallaoua, D., et al. Effects of isoproterenol and cholera toxin on human limbal epithelial cell cultures. Curr Eye Res 37, 644-653 (2012).
[8] Zhang, T., et al. The effect of amniotic membrane de-epithelialization method on its biological properties and ability to promote limbal epithelial cell culture. Invest Ophthalmol Vis Sci 54, 3072-3081 (2013).
[9] Gonzalez, S. & Deng, S. X. Presence of native limbal stromal cells increases the expansion efficiency of limbal stem/progenitor cells in culture. Exp Eye Res 116, 169-176 (2013).
[10] Di Iorio, E., et al. Q-FIHC: quantification of fluorescence immunohistochemistry to analyse p63 isoforms and cell cycle phases in human limbal stem cells. Microscopy research and technique 69, 983-991 (2006).
[11] Notman, R., Noro, M., O'Malley, B. & Anwar, J. Molecular basis for dimethylsulfoxide (DMSO) action on lipid membranes. J Am Chem Soc 128, 13982-13983 (2006).
[12] Windrum, P., et al. Variation in dimethyl sulfoxide use in stem cell transplantation: a survey of EBMT centres. Bone marrow transplantation 36, 601-603 (2005).
[13] Trosan, P., et al. The key role of insulin-like growth factor I in limbal stem cell differentiation and the corneal wound-healing process. Stem Cells Dev 21, 3341-3350 (2012).
[14] Wilson, S. E., et al. Effect of epidermal growth factor, hepatocyte growth factor, and keratinocyte growth factor, on proliferation, motility and differentiation of human corneal epithelial cells. Exp Eye Res 59, 665-678 (1994).
[15] Rheinwald, J. G. & Green, H. Epidermal growth factor and the multiplication of cultured human epidermal keratinocytes. Nature 265, 421-424 (1977).
[16] Miyashita, H., et al. Long-term maintenance of limbal epithelial progenitor cells using rho kinase inhibitor and keratinocyte growth factor. Stem Cells Transl Med 2, 758-765 (2013).
[17] Judd, et al. Defined systems for epithelial cell culture and use thereof. U.S. Pat. No. 6,692,961.

Further information on the present invention can be found in Gonzalez et al., Tissue Engineering Part C: Methods. April 2017, 23(4): 219-227 which is incorporated by reference herein. All publications mentioned herein are incorporated herein by reference to disclose and describe the methods and/or materials in connection with which the publications are cited. Publications cited herein are cited for their disclosure prior to the filing date of the present application. Nothing here is to be construed as an admission that the inventors are not entitled to antedate the publications by virtue of an earlier priority date or prior date of invention. Further, the actual publication dates may be different from those shown and require independent verification.

### Conclusion

This concludes the description of the preferred embodiment of the present invention. The foregoing description of one or more embodiments of the invention has been presented for the purposes of illustration and description. It is not intended to be exhaustive or to limit the invention to the precise form disclosed. Many modifications and variations are possible in light of the above teaching.

1. A human limbal epithelial stem cell culture media comprising:
isoproterenol;
Human Epidermal Growth Factor (EGF); and
an antibiotic;
wherein:
the media does not contain cholera toxin; and/or
the media does not contain dimethylsulfoxide (DMSO).

2. The cell culture media of claim 1, wherein the media comprises from 1% to 20% human serum (v/v).

3. The cell culture media of claim 1, wherein the media comprises at least one of:
from 0.5-2 μg/mL isoproterenol;
from 0.4-10 ng/mL Human Epidermal Growth Factor (EGF);
from 0.4-5 μg/mL hydrocortisone; and
at least one of penicillin, streptomycin, gentamicin or amphotericin B.

4. The cell culture media of claim 1, further comprising at least one of:
insulin;
transferrin;
selenite;
progesterone; and
putrescine.

5. The cell culture media of claim 1, wherein the media further comprises a denuded amniotic membrane.

6. The cell culture media of claim 1, wherein the media further comprises human limbal epithelial stem cells.

7. The cell culture media of claim 6, wherein the human limbal epithelial stem cells are disposed within a limbal tissue explant.

8. The cell culture media of claim 1, wherein the media is free of xenobiotic supplements.

9. The cell culture media of claim 1, wherein human limbal epithelial stem cells growing in the media comprise greater than 3% p62α bright cells.

10. The cell culture media of claim 1, wherein the media comprises not more than 0.4 ng/mL Human Epidermal Growth Factor (EGF).

11. A method of growing human limbal epithelial stem cells comprising disposing the cells in a cell culture media of claim 1 at a temperature and under CO₂ concentrations sufficient for the human limbal epithelial stem cells to grow.

12. The method of claim 11, wherein a preponderance of the human limbal epithelial stem cells do not differentiate after being disposed in the media.

13. The method of claim 12, wherein the differentiation state of the cell is observed by observing cell morphology.

14. The method of claim 12, wherein the differentiation state of the cell is observed by observing expression of a polypeptide in the human limbal epithelial stem cells.

15. The method of claim 11, wherein the temperature is between 35° C. to 38° C.

16. The method of claim 11, wherein the CO₂ concentration is between 4-10% CO₂.

17. The method of claim 11, wherein populations of human limbal epithelial stem cells growing in the media comprise greater than 3% p62α bright human limbal epithelial stem cells.

18. The method of claim 11, wherein the media comprises not more than 0.4 ng/mL Human Epidermal Growth Factor (EGF).

19. The method of claim 11, wherein the media is free of xenobiotic components.

20. A transport system for cultured limbal stem cells comprising:
a transport container;
a screw-cap that forms a tight seal with the transport container;
a ring attached to the cap adapted to stabilize the cLSCs; and
a cell transport media comprising:
DMEM/F12;
HEPES;
Human EGF;
Human AB serum;
Hydrocortisone;
Penicillin-Streptomycin;
Gentamicin; and
Amphotericin B;
wherein:
the media does not contain cholera toxin; and/or
the media does not contain dimethylsulfoxide (DMSO).
